(19) United States
(12) Patent Application Publication
(10) Pub. No.: US 2009/0111134 A1
(43) Pub. Date: Apr. 30, 2009

(54) Multiplex PCR Assay For Identification of USA300 and USA400 Community-Associated Methicillin Resistant Staphylococcal Aureus Strains

(76) Inventors: Kunyan Zhang, Calgary (CA); John Conly, Calgary (CA)

Correspondence Address: FULBRIGHT & JAWORSKI L.L.P., 600 CONGRESS AVE., SUITE 2400, AUSTIN, TX, 78701, US

(21) Appl. No.: 12/061,847

(22) Filed: Apr. 3, 2008

Related U.S. Application Data
(60) Provisional application No. 60/912,846, filed on Apr. 19, 2007.

(51) Int. Cl. C12Q 1/48

(52) U.S. Cl. 435/15

(57) ABSTRACT

The present invention relates to multiplex polymerase chain reaction (PCR) assays for Staphylococcus aureus typing. In particular, the invention relates to identification, detection and classification of USA300 and USA400 strains, the predominant community-associated methicillin-resistant S. aureus (MRSA) strains in North America, as well as other strains, and simultaneous detection of PVL genes and discrimination of MRSA from methicillin-susceptible S. aureus (MSSA) and S. aureus from coagulase-negative staphylococci (CoNS).

The present application claims benefit of priority to U.S. Provisional Application Ser. No. 60/912,846, filed Apr. 19, 2007, the entire contents of which are hereby incorporated by reference.

### BACKGROUND OF THE INVENTION

A. Field of the Invention

The present invention relates to the fields of microbiology, diagnostics and molecular biology. More specifically, a multiplex polymerase chain reaction assay for Staphylococcus aureus typing is disclosed that can be used to identify, detect and classify USA300 and USA400 strains (predominantly community-associated North American MRSA strains), and simultaneously detect PVL genes and discriminate MRSA from methicillin-susceptible S. aureus (MSSA) and S. aureus from coagulase-negative staphylococci (CoNS).

B. Related Art

Staphylococcus aureus is a major human pathogen, causing a wide variety of illnesses ranging from mild skin and soft tissue infections and food poisoning to life-threatening illnesses such as deep post-surgical infections, septicaemia, endocarditis, necrotizing pneumonia, and toxic shock syndrome. These organisms have a remarkable ability to accumulate additional antibiotic resistance determinants, resulting in the formation of multiply-drug-resistant strains. Methicillin, being the first semi-synthetic penicillin to be developed, was introduced in 1959 to overcome the problem of penicillin-resistant S. aureus due to β-lactamase (penicillinase) production (Livermore, 2000). However, methicillin-resistant S. aureus (MRSA) strains were identified soon after the introduction of methicillin (Barber, 1961; Jevons, 1961). MRSA have acquired and integrated into their genome a 21- to 67-kb mobile genetic element, termed the staphylococcal cassette chromosome mec (SCCmec) that harbors the methicillin resistance (mecA) gene and other antibiotic resistance determinants (Ito et al., 2001; Ito et al., 2004; Ma et al., 2002). The mecA gene encodes an altered additional low affinity penicillin-binding protein (PBP2a) that confers broad resistance to all penicillin-related compounds including cephalosporins and carbapenems that are currently some of the most potent broad-spectrum drugs available (Hackbarth & Chambers, 1989). Since their first identification, strains of MRSA have spread and become established as major nosocomial (hospital-acquired (HA)-MRSA) pathogens worldwide (Ayliffe, 1997; Crossley et al., 1979; Panlilio et al., 1992; Voss et al., 1994). Recently, these organisms have evolved and emerged as a major cause of community-acquired infections (CA-MRSA) in healthy individuals lacking traditional risk factors for infection, and are causing community-outbreaks, which pose a significant threat to public health (Begier et al., 2004; Beilman et al., 2005; Conly et al., 2005; Gilbert et al., 2006; Gilbert et al., 2005; Harbarth et al., 2005; Holmes et al., 2005; Issartel et al., 2005; Ma et al., 2005; Mulvey et al., 2005; Robert, Etienne & Bertrand, 2005; Said-Salim et al., 2005; Vandenesch et al., 2003; Vourli et al., 2005; Wannet et al., 2005; Wannet et al., 2004; Witte et al., 2005; Wylie & Nowicki, 2005).

The incidence of MRSA infection has greatly increased over the past 5 years due to the spread of community-associated MRSA. The two predominant strains of CA-MRSA circulating in North America belong to pulsed-field gel types USA300 and USA400 strains according to the CDC classification. The USA300 and USA400 stains have been associated with serious infections including soft tissue abscesses, cellulitis, necrotizing fasciitis, severe multifocal osteomyelitis, bacteremia with Waterhouse-Frederickson syndrome, septic shock and necrotizing pneumonia (Beilman et al., 2005; CDC, 2003; Conly et al., 2005; Francis et al., 2005; Kazakova et al., 2005). Of greater concern is the high transmissibility of USA300 and the link between both USA300 and USA400 and disease outbreaks worldwide (Kazakova et al., 2005; Pan et al., 2003; Tenover et al., 2006). Another alarming observation is that community-associated MRSA strains, in particular USA300, are being reported as causing hospital acquired MRSA infections as well (Bratu et al., 2005; Chalumeau et al., 2005; Linde et al., 2005; Naas et al., 2005; Perdreau-Remington et al., 2004).

The USA400 strain is represented by strain MW2, isolated in 1998 in North Dakota from a pediatric patient with fatal septicaemia (1999). The MW2 genome has been fully sequenced and shown to contain 4 genomic islands (νSa3, νSa4, νSaα and νSaβ) 2 prophages (φSa2mw and φSa3mw) and an SCCmec element (IVa), all of which contribute to its virulence (Baba et al., 2002). MW2 is a hypervirulent strain carrying a large number of toxin genes, including new allelic forms of enterotoxins L (sel2) and C (sec4) on νSa3, 11 putative exotoxins (set16-26) on νSaα, lukD and lukE leukotoxins on νSaβ, enterotoxin A (sea), Q (seq) and 2 new allelic forms of enterotoxin G (seg2) and K (sek2) on prophage φSa3mw (Baba et al., 2002). Prophage φSa2mw harbours the lukS-PV and the lukF-PV genes, encoding the PVL components (Baba et al., 2002). Also found in the MW2 genome, but not associated with genomic islands or prophages, are the genes encoding γ-hemolysin (hlg) and enterotoxin H (seh) (Baba et al., 2002).

The USA300 strain, represented by strain FPR3757, was isolated in 2000 from an inmate in a California prison (2001). It has been sequenced and similar to USA400, found to contain multiple genetic elements which contribute to virulence, including an SCCmec element (IVa), 2 prophages (φSa2usa and φSa3usa), 3 pathogenicity islands (SaPI5, νSaα and νSaβ) and the Arginine Catabolic Mobile Element (ACME) (Diep et al., 2006). In contrast to the USA400 genome, which bears a large number of toxin genes, the genome of USA300 carries a smaller number of toxin genes, including enterotoxins K and Q on SaPI5 and set30-39 on νSaα. Prophage φSa2usa is very similar in structure to φSa2mw and, likewise, carries the PVL genes, lukS-PV and lukF-PV. Unique to the USA300 genome is the presence of a 30.9 kb ACME complex. The ACME complex is integrated into the chromosome at the same attachment site as SCCmec and contains an arc gene cluster, encoding an arginine deiminase pathway, as well as a putative oligopeptide permease operon, Opp (Diep et al., 2006). In addition to USA300 strain, the ACME complex has been found in Staphylococcus capitis and Staphylococcus epidermidis, but due to its high frequency of occurrence in S. epidermidis it is believed to have transferred to USA300 from this species (Diep et al., 2006).

The USA300 and USA400 strains belong to multi-locus sequence typing (MLST) type 8 (ST8) and ST1, respectively and both carry Panton-Valentine leukocidin (PVL) genes and SCCmec type IVa. To date, there is no rapid way to identify and characterize CA-MRSA, but rather numerous times and labor intensive molecular characterization tests. An accurate and rapid PCR based assay, able to distinguish USA300 and USA400 isolates from other MRSA, would facilitate in the identification of outbreaks, treatment of patients and aid in the implementation of control measures designed to limit the spread of these serious pathogens.

### SUMMARY OF THE INVENTION

To address the above-noted shortcomings in the filed, the present inventors have designed a multiplex PCR (M-PCR) assay capable of accurately identifying USA300 and USA400 strains, and simultaneously detecting PVL genes and discrimination of MRSA from methicillin-susceptible S. aureus (MSSA) and S. aureus from CoNS.

Thus, in accordance with the present invention, there is provided a method for identifying a methicillin-resistant Staphylococcal aureus (MRSA) bacterium in a sample comprising (a) obtaining a biological sample suspected of containing a MRSA bacterium; (b) subjecting nucleic acids from said sample to multiplex PCR targeting the following genes: 16s rRNA, arcA, lukS/F-PV, MW756, nuc, MW1409, mecA and MW1438; (c) detecting amplification products resulting from said multiplex PCR; and (d) comparing the detected amplification products from step (c) with those predicted for known MRSA and/or non-MRSA bacteria, wherein an amplification product profile similar or identical to that observed for known MRSA bacteria identifies said sample as comprising a MRSA bacterium. The assay may further discriminate between USA300/USA400 and other MRSA strains, and/or between USA300 and USA400.

The biological sample may be a clinical sample, such as urine, blood, sputum, saliva, or pus. The biological sample may be a culture sample, such as from a broth, a plate or membrane. Blood culture and culture on blood agar are specifically contemplated The multiplex PCR may be performed using one or more of the following primer pairs: SEQ ID NO:1 and SEQ ID NO:2; SEQ ID NO:3 and SEQ ID NO:4; SEQ ID NO:5 and SEQ ID NO:6; SEQ ID NO:7 and SEQ ID NO:8; SEQ ID NO:9 and SEQ ID NO:10; SEQ ID NO:11 and SEQ ID NO:12; SEQ ID NO:13 and SEQ ID NO:14; and/or SEQ ID NO:15 and SEQ ID NO:16, and the multiplex PCR may be performed using each of these primer pairs. The detection of amplification products may comprise electrophoretic separation and visualization of separated amplification products.

In another embodiment, there is provided a kit comprising primer pairs for amplifying the following genes: 16s rRNA, arcA, lukS/F-PV, MW756, nuc, MW1409, mecA and MW1438, in one or more suitable containers. The primer pairs may, in particular, be: SEQ ID NO:1 and SEQ ID NO:2; SEQ ID NO:3 and SEQ ID NO:4; SEQ ID NO:5 and SEQ ID NO:6; SEQ ID NO:7 and SEQ ID NO:8; SEQ ID NO:9 and SEQ ID NO:10; SEQ ID NO:11 and SEQ ID NO:12; SEQ ID NO:13 and SEQ ID NO:14; and SEQ ID NO:15 and SEQ ID NO:16. The kit may further comprise a buffer, diluent and/or excipient in at least one additional suitable container, may also further comprise a enzyme that directs polymerase chain reaction in at least one additional suitable container, and may also further comprising at least one molecular weight standard or PCR substrate standard in at least one additional suitable container.

It is contemplated that any method or composition described herein can be implemented with respect to any other method or composition described herein.

The use of the word, “a” or “an” when used with the term “comprising” in the specification and/or claims may mean “one,” “one or more,” “at least one,” or “one or more than one.”

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-B—Molecular, Genomic and antimicrobial susceptibility profiles of well-representative control strains. (FIG. 1A) highlights the utility of PFGE (pulsed field gel electrophoresis), PVL (Panton Valentine leukocidin) genes, SCCmec (staphylococcal cassette chromosome mec) typing, spa (staphylococcal protein A) typing, MLST (multilocus sequence typing), and antimicrobial resistance phenotypes for strain characterization. (FIG. 1B) eBURST analysis to compare our MLST data (15 ST types) with International MLST database (no. isolates=1695; no. STs=540; No. groups=40; updated October 2006). The MLST data was divided into groups of related clonal complexes, the founding genotype of each clonal complex was predicted, and the bootstrap support for the assignment was computed.

FIGS. 2A-C—Single target PCR from the representative isolates showed specificity for the strain- and the phage-specific new sets of primers. (FIG. 2A) φSa2mw/φSa2usa phage-specific primers phi-int-F4 and phi-int-R4 target the gene MW1409; (FIG. 2B) USA400 strain-specific primers MW756-F and MW756-R target the gene MW756 on the genomic island νSa3 of MW2; (FIG. 2C) USA300 strain-specific primers arcA-F and arcA-R target the arcA gene on the arginine catabolic mobile element (ACME) island. Lane 1, Canadian epidemic CA-MRSA USA400 control strain CMRSA-7 (PVL+; φSa2mw/φSa2usa+; MW756+; arcA−); lane 2, PVL (−) USA400 strain C2901 (PVL−; φSa2mw/φSa2usa+; MW756+; arcA−); lane 3, Canadian epidemic CA-MRSA USA300 control strain CMRSA-10 (PVL+; φSa2mw/φSa2usa+; MW756−; arcA+); lane 4, Canadian epidemic HA-MRSA control strain CMRSA-2 (PVL−; φSa2mw/φSa2usa−; MW756−; arcA−); lane 5, PVL (−) MR-CoNS strain CNS99-PF6 (PVL−; φSa2mw/φSa2usa−; MW756−; arcA−); lane 6, PVL (−) MR-CoNS strain CNS99-PF8 (PVL−; φSa2mw/φSa2usa−; MW756−; arcA+); lane M, 1 Kb Plus DNA Ladder (Invitrogen). Arrows indicating the corresponding PCR products. Refer to FIGS. 1A-B and Table 2 for details of each strain.

FIG. 3-Novel multiplex PCR assay identifies USA300 and USA400 community-associated MRSA strains, detects Panton-Valentine Leukocidin (PVL) and mecA Genes, and simultaneously discriminates S. aureus from Coagulase-Negative Staphylococci (CoNS). Lane 1, strain ATCC 29213 (PVL− MSSA); lane 2, strain ATCC 49775 (PVL+ MSSA); lane 3, Canadian epidemic HA-MRSA control strain CMRSA-2 (PVL− non-USA300 and non-USA400 MRSA); lane 4, strain C1538 (PVL+ non-USA300 and non-USA400 MRSA); lane 5, strain C2901 (PVL− USA400); lane 6, Canadian epidemic CA-MRSA USA400 control strain CMRSA-7 (PVL+ USA400); lane 7, Canadian epidemic CA-MRSA USA300 control strain CMRSA-10 (PVL+ USA300); lane 8, strain CNS99-PF5 (PVL− & arcA− MS-CoNS); lane 9, strain CNS99-PF7 (PVL− but arcA+ MS-CoNS); lane 10, strain CNS99-PF6 (PVL− & arcA− MR-CoNS); lane 11, strain CNS99-PF8 (PVL− but arcA+ MR-CoNS); lane M, 1 Kb Plus DNA Ladder (Invitrogen). Refer to FIGS. 1A-B and Table 2 for details of each strain.

FIG. 4-Shared genotypic and phenotypic traits of PVL(+) USA300 and PVL(+)/PVL(−) USA400 clinical isolates. Only a single representative isolate from individual pulsed field gel electrophoresis (PFGE) clonotypes is shown. The solid boxes delineate the groups of USA300 and USA400 strains with minor PFGE variations (patterns A, B, C as indicated in brackets). CMRSA-10, Canadian epidemic CA-MRSA USA300 control strain; CMRSA-7, Canadian epidemic CA-MRSA USA400 control strain; Isolate, local clinical isolates of USA300 and USA400 strains collected and tested in this study; PVL, Panton-Valentine Leukocidin (+, positive; −, negative); SCCmec, staphylococcal cassette chromosome mec; agr, accessory gene regulator; spa (staphylococcal protein A gene) motif: t008 (YHGFMBQBLO) and t128 (UJJFKBPE); MLST (multilocus sequence typing) profile: ST8 (3-3-1-1-4-4-3) and ST1 (1-1-1-1-1-1-1); Pen, penicillin; Oxa, Oxacillin; Ery, Erythromycin; Clin, Clindamycin; Gen, Gentamicin; Cip, Ciprofloxacin; Tet, Tetracycline; Rif, Rifampin; SXT, trimethoprin-sulfamethoxazole; Van, Vancomycin; S, susceptible; R, resistant (with resistant rate in brackets).

### DETAILED DESCRIPTION OF PREFERRED EMBODIMENTS

### A. The Present Invention

The emerging spread of CA-MRSA strains poses a significant threat to public health (Lindsay, 2004; Vandenesch, 2003). USA300 and USA400 are the predominant CA-MRSA strains circulating in North America, implicated in outbreaks of community-onset infections, leading to significant morbidity and mortality. The USA300 and USA400 strains belong to multi-locus sequence typing (MLST) type 8 (ST8) and ST1, respectively, and both carry PVL genes and SCCmec type IVa. However, molecular characterization of these strains can be time consuming and technically laborious. The inventors have developed a new multiplex PCR assay for the identification of the USA300 and USA400 strains, and simultaneous detection of PVL genes and discrimination of MRSA from MS SA and S. aureus from CoNS.

1. Samples

Samples for testing may come from a variety of sources, and may be known to contain MRSA strains of undetermined genetic make-up, known to contain bacteria of undetermined MRSA status, or simply suspected of containing bacteria. As such, the assays of the present invention may be use for simple detection, classification of MRSA versus non-MRSA, or classification within the MRSA categories.

The sample may come directly from a subject, for example, by the obtaining of a clinical sample, including blood, urine, saliva, mucous, pus, sputum, lavage (tracheal, bronchial, gastric), douches, enemas, etc. The sample may be obtained through direct withdrawal from a patient site using a probe (swab, needle, catheter, gauze or membrane), or may be obtained off a medical device following an ancillary procedure (bandage, needle, syringe, suction line).

Alternatively, the sample may be the result of culturing of a primary isolate from a patient or other source (such as a contaminated device or nutrient source). This is particularly useful where very low, and possibly undetectable amounts, of bacterial nucleic acid are present. The primary isolate will be used to inoculate a culture such as a broth (tube or flask) or plate (agar) which is then incubated at a permissive temperatures (e.g., 37° C.). After sufficient time, the resulting bacteria, if any are either tested directly or isolated for clonal expansion, after which analysis is performed. Blood culture and culture on blood agar are specifically contemplated. Culture times may vary from several hours (6, 8, 10, 12, 12-24 hrs) to several days (1, 2, 3 or 4 days).

2. Multiplex PCR

As used herein, “polymerase chain reaction” or “PCR” is a molecular biology technique for enzymatically replicating DNA without using a living organism. The technique allows for small amount of the DNA molecule to be amplified many times, in an exponential manner, with more DNA templates available after every cycle.

PCR, as currently practiced, requires several basic components. These components are (a) DNA template that contains the region of the DNA fragment to be amplified; (b) one or more primers, which are complementary to the DNA regions at the 5′ and 3′ ends of the DNA region that is to be amplified; (c) a DNA polymerase (e.g., Taq polymerase or another DNA polymerase with a temperature optimum at around 70° C.), used to synthesize a DNA copy of the region to be amplified; (d) deoxynucleotide triphosphates, (dNTPs) from which the DNA polymerase builds the new DNA; (e) buffer solution, which provides a suitable chemical environment for optimum activity and stability of the DNA polymerase; (f) divalent cation, magnesium or manganese ions; generally Mg²⁺ is used, but Mn²⁺ can be utilized for PCR-mediated DNA mutagenesis, as higher Mn²⁺ concentration increases the error rate during DNA synthesis; and monovalent cation potassium ions. The PCR is carried out in small reaction tubes (0.2-0.5 ml volumes), containing a reaction volume typically of 15-100 μl, that are inserted into a thermal cycler. This is a machine that heats and cools the reaction tubes within it to the precise temperature required for each step of the reaction. Most thermal cyclers have heated lids to prevent condensation on the inside of the reaction tube caps. Alternatively, a layer of oil may be placed on the reaction mixture to prevent evaporation.

As used herein, a “multiplex polymerase chain reaction” or “multiplex PCR” is a PCR reaction where more than one primer set is included in the reaction pool allowing two or more different targets to be amplified by PCR in a single reaction tube. It is important that the various primers be specific for their target genes and be used under conditions that prevent cross-priming of non-target sequences.

Oligonucleotide synthesis is well known to those of skill in the art. Various mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference in its entirety. Basically, chemical synthesis can be achieved by the diester method, the triester method polynucleotides phosphorylase method and by solid-phase chemistry.

The diester method was the first to be developed to a usable state, primarily by Khorana and co-workers (Khorana, 1979). The basic step is the joining of two suitably protected deoxynucleotides to form a dideoxynucleotide containing a phosphodiester bond. The diester method is well established and has been used to synthesize DNA molecules (Khorana, 1979).

The main difference between the diester and triester methods is the presence in the latter of an extra protecting group on the phosphate atoms of the reactants and products (Itakura et al., 1975). The phosphate protecting group is usually a chlorophenyl group, which renders the nucleotides and polynucleotide intermediates soluble in organic solvents. Therefore, purifications are done in chloroform solutions. Other improvements in the method include (i) the block coupling of trimers and larger oligomers, (ii) the extensive use of high-performance liquid chromatography for the purification of both intermediate and final products, and (iii) solid-phase synthesis.

This is an enzymatic method of DNA synthesis that can be used to synthesize many useful oligodeoxynucleotides (Gillam et al., 1978). Under controlled conditions, polynucleotide phosphorylase adds predominantly a single nucleotide to a short oligodeoxynucleotide. Chromatographic purification allows the desired single adduct to be obtained. At least a trimer is required to initiate the method of adding one base at a time, a primer that must be obtained by some other method. The polynucleotide phosphorylase method works and has the advantage that the procedures involved are familiar to most biochemists.

The technology developed for the solid-phase synthesis of polypeptides has been applied after an, it has been possible to attach the initial nucleotide to solid support material has been attached by proceeding with the stepwise addition of nucleotides. All mixing and washing steps are simplified, and the procedure becomes amenable to automation. These syntheses are now routinely carried out using automatic DNA synthesizers.

Phosphoramidite chemistry (Beaucage, 1993) has become by far the most widely used coupling chemistry for the synthesis of oligonucleotides. As is well known to those skilled in the art, phosphoramidite synthesis of oligonucleotides involves activation of nucleoside phosphoramidite monomer precursors by reaction with an activating agent to form activated intermediates, followed by sequential addition of the activated intermediates to the growing oligonucleotide chain (generally anchored at one end to a suitable solid support) to form the oligonucleotide product.

Taq polymerase (“Taq Pol,” or simply “Taq”) is a thermostable polymerase used in polymerase chain reaction to check for the presence or absence of a gene by amplifying a DNA fragment. It replaced E. coli DNA polymerase in PCR because of the temperature conditions of PCR. First isolated from Thermus aquaticus (hence the abbreviation “Taq”), a bacterium that lives in hot springs and hydrothermal vents, Taq was identified as the first polymerase able to withstand the denaturing conditions required during PCR. Its enzymatic halflife (at 95° C.) is 40 min. One of Taq polymerases' drawbacks is its low replication fidelity since it lacks a 3′ to 5′ exonuclease proofreading mechanism. Commercially sold Taq DNA polymerase has an error rate of one in 10,000 nucleotides and typically produces 16% of mutated 1 kb PCR products in a reaction. It can amplify a 1 kb strand of DNA in roughly 30 seconds at 72° C. Despite its error rate, Taq DNA polymerase can still be used in experiments where an identical genetic sequence is required (such as in molecular cloning).

3. Separation of Amplification Products

In certain embodiments, nucleic acid products are separated by agarose, agarose-acrylamide or polyacrylamide gel electrophoresis using standard methods (Sambrook et al., 1989). Separation of nucleic acids may also be effected by chromatographic techniques known in the art. There are many kinds of chromatography that may be used in the practice of the present invention, including capillary adsorption, partition, ion-exchange, hydroxylapatite, molecular sieve, reverse-phase, column, paper, thin-layer, and gas chromatography as well as HPLC.

A number of the above separation platforms can be coupled to achieve separations based on two different properties. For example, some of the primers can be coupled with a moiety that allows affinity capture, and some primers remain unmodified. Modifications can include a sugar (for binding to a lectin column), a hydrophobic group (for binding to a reverse-phase column), biotin (for binding to a streptavidin column), or an antigen (for binding to an antibody column). Samples are run through an affinity chromatography column. The flow-through fraction is collected, and the bound fraction eluted (by chemical cleavage, salt elution, etc.). Each sample is then further fractionated based on a property, such as mass, to identify individual components.

Nucleic acids may be visualized in order to confirm their presence, quantity or sequence. In one embodiment, the primer is conjugated to a chromophore, radiolabel or fluorometric label. In another embodiment, the primer is conjugated to a binding partner that carries a detectable moiety, such as an antibody or biotin. In other embodiments, the primer incorporates a fluorescent dye or label. In yet other embodiments, the primer has a mass label that can be used to detect the molecule amplified. Other embodiments also contemplate the use of Taqman™ and Molecular Beacon™ probes. Alternatively, one or more of the dNTPs may be labeled with a radioisotope, a fluorophore, a chromophore, a dye or an enzyme. Also, chemicals whose properties change in the presence of DNA can be used for detection purposes. For example, the methods may involve staining of a gel with, or incorporation into the separation media, a fluorescent dye, such as ethidium bromide or Vistra Green, and visualization under an appropriate light source.

4. Reference Strains

Reference strains are described in the Materials and Methods section of the Examples, below.

### B. Examples

The examples below are carried out using standard techniques, which are well known and routine to those skilled in the art. These examples are intended to be illustrative, but not limiting, of the invention.

### Example 1

### Materials and Methods

Bacterial strains and isolates. The Canadian epidemic MRSA reference strains, CMRSA-1 to 10, and strain N02-590 were provided by National Microbiology Laboratory, Health Canada, Winnipeg, Canada (Mulvey et al., 2005; Simor et al., 2002). Local control strains of MRSA and MSSA were from the inventors' collection and had previously undergone phenotypic and genotypic (pulsed-field gel electrophoresis [PFGE], staphylococcal protein A [spa] typing, and multilocus sequence typing [MLST]) analyses (below). Clinical isolates of MRSA, MSSA and CoNS used for validation of the multiplex-PCR (M-PCR) assay were randomly selected from the Calgary Laboratory Services (CLS) frozen clinical isolate stock collected over the August 1999 to January 2006 time period. Additional historical clinical MRSA and MSSA strains were recovered from 5 tertiary acute-care teaching hospitals located in 4 cities in 3 provinces of the Canadian Prairies (Winnipeg, Manitoba; Saskatoon, Saskatchewan; Calgary, Alberta; and Edmonton, Alberta) during the 1989-1994 period (Embil et al., 1994).

Identification and phenotypic susceptibility testing of staphylococcal isolates. The staphylococcal isolates were identified morphologically and biochemically by standard laboratory procedures (Murray, 2003). The coagulase plasma test (Remel, Lenexa, Kans., USA) was performed on organisms exhibiting typical staphylococcal colony morphology to allow for discrimination of S. aureus from coagulase-negative staphylococci (CoNS). Screening for methicillin and other antibiotic resistance phenotypes was done by VITEK (bioMerieux, Inc. Durham, N.C., USA) along with the CLSI oxacillin agar screen, while confirmation of methicillin resistance was achieved using an in-house assay for the mecA gene (Hussain et al., 2000).

Molecular and genomic characterization of isolates. Isolates were further tested for confirmation of methicillin resistance (the mecA gene) and for the presence of PVL (lukS-P V and lukF-PV genes) with a triplex PCR assay (McClure et al., 2006). Isolates were genetically typed using PFGE after digestion with SmaI following a standardized protocol (Mulvey et al., 2001). PFGE-generated DNA fingerprints were digitized and analyzed with BioNumerics Ver. 3.5 (Applied Maths, Sint-Martens-Lattem, Belgium) by using a position tolerance of 1.0 and an optimization of 1.0. Cluster analysis was performed by the un-weighted pair group method, using arithmetic averages (UPGMA), and DNA relatedness was calculated on the basis of the Dice coefficient. Isolates were considered to be genetically related if their macrorestriction DNA patterns differed by <7 bands and the Dice coefficient of correlation was >75% (Simor et al., 2002; Tenover et al., 1995). The MRSA isolates were typed for staphylococcal cassette chromosome mec (SCCmec) using a multiplex PCR SCCmec typing assay to classify the isolates as type and subtypes I, II, III, IVa, IVb, IVc, IVd, and V (Zhang et al., 2005). Staphylococcal protein A (spa) (Harmsen et al., 2003; Shopsin et al., 1999) and MLST (Enright et al., 2000) typing were conducted on representative isolates as previously described. The clonal complex (CC) analysis of the control strains was conducted through comparative eBURST analysis by comparison of the control strain MLST database with the international S. aureus MLST dataset available via the MLST Home Page (www.mlst.net; updated as of October 2006), using the eBURSTv3 program (Feil et al., 2004; Spratt et al., 2004). The identification of MRSA isolates matching the USA300 and USA400 CA-MRSA strains was based on the similarity of PFGE patterns to the USA300 and USA400 control strains and the presence of PVL, SCCmec type IVa, spa type t008, and MLST type ST8 for USA300 and PVL, SCCmec type IVa, spa type t128, and MLST type ST1 for USA400.

Sequence alignment and primer design. Primers for 16S rRNA (Staphylococcus genus-specific) (Zhang et al., 2004), nuc (S. aureus species-specific) (Shortle, 1983; Zhang et al., 2004), mecA (a determinant of methicillin resistance) (Zhang et al., 2005) and lukS/F-PV (PVL genes) (Lina et al., 1999; McClure et al., 2006) were as previously described (Table 1). New sets of specific primers for CA-MRSA strains USA300 and USA400 and for prophage (φSa2usa from USA300 and φSa2mw from USA400), were designed based on a comprehensive analysis and alignment of individual Staphylococcus sp. genomes currently available in the GenBank database (National Center for Biotechnology Information, USA; updated as of July 2006). Gene targets and specificity for each primer pair are as follows: USA300 strain-specific primers arcA-F and arcA-R target the arcA gene (coding for arginine deiminase, the central enzyme in the complete arginine deiminase pathway) on the arginine catabolic mobile element (ACME) island; USA400 strain specific primers MW756-F and MW756-R target the gene MW756 (encoding a hypothetical protein) on the genomic island νSa3 of MW2; φSa2mw and φSa2usa prophage specific primers phi-int-F4 and phi-int-R4 target the gene MW1409 (encoding a hypothetical protein) on the prophage φSa2mw of USA400 strain MW2 or SAUSA300_{—}1410 (encoding a virulence-associated protein E) and SAUSA300_{—}1411 (encoding a phiSLT ORF66-like protein) on the prophage φSa2usa of USA300 strain FPR3757. Details of primer sequences, gene targets, specificity, concentration, amplicon size and accession numbers are listed in Table 1. The oligonucleotide primers used in this study were synthesized and purchased from University of Calgary Core DNA Services Laboratory (University of Calgary, Calgary, Canada).

DNA extraction. Frozen bacteria were subcultured twice onto Tryptic soy agar (TSA) plates (Becton Dickinson, Sparks, Md., USA) prior to DNA extraction. One to five bacterial colonies were suspended in 50 μl of sterile distilled water and heated at 95° C. for 10 min. After centrifugation at 30,000×g (13,000 rpm) for 1 min, 2 μl of the supernatant was used as template in a 25 μl PCR reaction (Zhang et al., 2004).

PCR amplification. This M-PCR assay contained 7 sets of primers targeted to the staphylococcal genes of 16S rRNA, arcA, lukS/F-PV, MW756, nuc, MW1409/SAUSA300_{—}1410-1411 and mecA. These primers and their respective concentrations used in the PCR are listed in Table 1. All PCR assays were performed using the rapid DNA isolation method described above. An aliquot of 4.65 μl of this suspension was added to 20.35 μl of PCR mixture containing 50 mM KCl, 20 mM Tris-HCl (pH 8.4), 2.5 mM MgCl₂, 0.2 mM of each dNTP (dATP, dTTP, dGTP, dCTP) (Invitrogen Inc., Carlsbad, Calif., USA), variable concentrations of the respective primers (Table 1), and 1.0 unit of Taq DNA polymerase (Invitrogen Inc., Carlsbad, Calif., USA). The amplification was performed in a GeneAmp PCR system 2720 or 9600 Thermal Cycler (Applied Biosystems, Foster City, Calif., USA), with the thermocycling conditions set at 94° C. for 4 min, followed by 10 cycles of 94° C. for 30 s, 60° C. for 30 s, 72° C. for 45 s, and another 25 cycles of 94° C. for 30 s, 52° C. for 30 s, and 72° C. for 45 s, ending with a final extension step at 72° C. for 10 min followed by a hold at 4° C. Single target PCR was conducted in a 25 μl reaction as above, but containing 0.2 μM of each primer, with the cycling parameters set at 94° C. for 4 min, followed by 30-35 cycles of 94° C. for 1 min, 50° C. for 1 min, and 72° C. for 2 min, ending with a final extension step at 72° C. for 10 min. The PCR amplicons were visualized using a UV light box after electrophoresis on a 2% agarose gel containing 0.5 μg/ml ethidium bromide.

Limiting dilution experiments for estimation of M-PCR sensitivity. The sensitivity of amplification of each primer pair in single target PCR and M-PCR was estimated by limiting dilution experiments (Zhang et al., 2004). Bacterial isolates were cultured on TSA plates overnight at 37° C. followed by suspension of colonies in sterile saline to a 1.0 McFarland turbidity standard. Ten-fold serial dilutions were made, after which DNA extraction was performed as described above. A 4.65 μl volume of DNA extract was used as the template in either the single target PCR or M-PCR as described above. The lower limits of detection (or minimal numbers of CFU detectable) of the target genes by single target PCR or M-PCR were then calculated based on correlation of the 1.0 McFarland standard to 3×10⁸ CFU/ml.

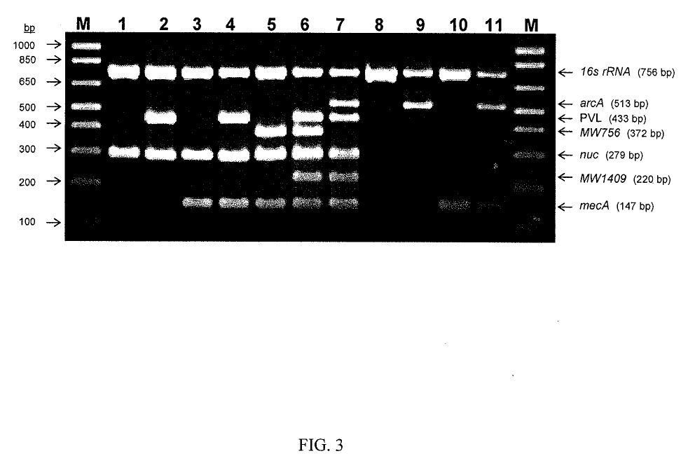

Validation and applicability of M-PCR detection method. The M-PCR assay was first optimized in 11 representative control strains (FIG. 3) and then validated by comparison with 48 well-characterized strains that had previously undergone detailed phenotypic and genotypic analyses (Table 2). The assay was subsequently applied to test a total of 1133 local clinical isolates belonging to several clonal groups and randomly selected from our clinical isolate frozen stock collection for the 1989-2006 time period (Table 4).

TABLE 1
Primers used in the USA300/USA400 Assay

| | | | | | | | |
|---|---|---|---|---|---|---|---|
| | Oligonucleotide | Conc. | Amplicon | | | GenBank | |
| Primer | Sequence (5′- 3′) | (μM) | size (bp) | Target | Specificity | # | Reference |

| | | | | | | | |
|---|---|---|---|---|---|---|---|
| Staph756-F | AACTCTGTTATTAGGGAAGAACA | 0.3 | 756 | 16S rRNA | Staphylo- | D83355 | Zhang, 2004 |
| | (SEQ ID NO:1) | | | | coccus | | |
| Staph750-R | CCACCTTCCTCCGGTTTGTCACC | | | | genus | | |
| | (SEQ ID NO:2) | | | | | | |
| arcA-F | GCAGCAGAATCTATTACTGAGCC | 0.3 | 513 | arcA | ACME | CP000255 | This study |
| | (SEQ ID NO:3) | | | | (USA300) | | |
| arcA-R | TGCTAACTTTTCTATTGCTTGAGC | | | | | | |
| | (SEQ ID NO:4) | | | | | | |
| Luk-PV-1 | ATCATTAGGTAAAATGTCTGGACATGATCCA | 0.4 | 433 | lukS/F-PV | PVL | X72700 | McClure, |
| | (SEQ ID NO:5) | | | | | | 2006; Lina, |
| Luk-PV-2 | GCATCAAGTGTATTGGATAGCAAAAGC | | | | | | 1999 |
| | (SEQ ID NO:6) | | | | | | |
| MW756-F | TGGTTAGCTATGAATGTAGTTGC | 0.32 | 372 | MW756 | Genomic | BA000033 | This study |
| | (SEQ ID NO:7) | | | | island | | |
| MW756-R | GTCCATCCTCTGTAAATTTTGC | | | | ν Sa3 | | |
| | (SEQ ID NO:8) | | | | (USA400) | | |
| Nuc-1 | GCGATTGATGGTGATACGGTT | 0.16 | 279 | nuc | S. aureus | V01281 | Zhand, |
| | (SEQ ID NO:9) | | | | species | | 2004; |
| Nuc-2 | AGCCAAGCCTTGACGAACTAAAGC | | | | | | Shortle, |
| | (SEQ ID NO:10) | | | | | | 1983 |
| phi-int-F4 | CAAATTTTGAAAACTTTACGC | 1.17 | 220 | MW1409 | Phages | BA000033; | This study |
| | (SEQ ID NO:11) | | | (USA400); | φSa2mw | CP000255 | |
| phi-int-R4 | TCCAGGATTAAAAGAAGCG | | | SAUSA300_ | (USA400) & | | |
| | (SEQ ID NO:12) | | | 1410 & 1411 | φSa2usa | | |
| | | | | (USA300) | (USA300) | | |
| mecA147-F | GTGAAGATATACCAAGTGATT | 0.16 | 147 | mecA | Methicillin | SAMECARI | Zhang, 2005 |
| | (SEQ ID NO:13) | | | | resistance | | |
| mecA147-R | ATGCGCTATAGATTGAAAGGAT | | | | | | |
| | (SEQ ID NO:14) | | | | | | |
| phi-F | GAAAAAAGTAATCGGACTGC | 0.36 | 426 | MW1438 | Phages | BA000033; | This study |
| | (SEQ ID NO:15) | | | (USA400); | φSa2mw | CP000255 | |
| | | | | SAUSA300_ | (USA400) & | | |
| | | | | 1436 | φSa2usa | | |
| | | | | (USA300) | (USA300) | | |
| phi-F | GAAAAAAGTAATCGGACTGC | 0.36 | 426 | MW1438 | Phages | BA000033; | This study |
| | (SEQ ID NO:16) | | | (USA400); | φSa2mw | CP000255 | |
| | | | | SAUSA300_ | (USA400) & | | |
| | | | | 1436 | φSa2usa | | |
| | | | | (USA300) | (USA300) | | |

TABLE 2
Molecular and genotypic features of S. aureus and CoNS control strains

| | | | | | | | | Genotype |
|---|---|---|---|---|---|---|---|---|
| | | | | | | | | Staph. sp. |
| Strain | | | Molecular | | SCC | SPA | ST | Specific |
| Type | Strain | PFGE Type | Type | PVL | mec | Type | Type | 16srRNA |
| PVL(+) | CMRSA10 | USA300-0114 | ST8-MRSA-IVa | + | IVa | t008 | ST8 | + |
| USA300 | | | | | | | | |
| | PMRSA-3 | USA300-0114 | ST8-MRSA-IVa | + | IVa | t008 | ST8 | + |
| | PMRSA-13 | USA300-0114 | ST8-MRSA-IVa | + | IVa | t008 | new | + |
| | PMRSA-16 | USA300-0114 | ST8-MRSA-IVa | + | IVa | t008 | ST8 | + |
| | PMRSA-46 | USA300-0114 | ST8-MRSA-IVa | + | IVa | t008 | ST8 | + |
| | PMRSA-12 | USA300 | ST8-MRSA-IVa | + | IVa | t008 | ST8 | + |
| PVL(−) | C2901 | USA400 | ST1-MRSA-IVa | − | IVa | t128 | ST1 | + |
| USA400 | | | | | | | | |
| | C2140 | USA400 | ST1-MRSA-IVa | − | IVa | t128 | ST1 | + |
| | C6022 | USA400 | ST1-MRSA-IVa | − | IVa | t128 | ST1 | + |
| PVL(+) | CMRSA-7 | USA400 | ST1-MRSA-IVa | + | IVa | t128 | ST1 | + |
| USA400 | | | | | | | | |
| | PMRSA-18 | USA400 | ST1-MRSA-IVa | + | IVa | t128 | ST1 | + |
| | PMRSA-50 | USA400 | ST1-MRSA-IVa | + | IVa | t128 | ST1 | + |
| | C10687 | USA400 | ST1-MRSA-IVa | + | IVa | t128 | ST1 | + |
| PVL(−) | CMRSA-1 | USA600 | ST45-MRSA-II | − | II | t004 | ST45 | + |
| MRSA | | | | | | | | |
| | CMRSA-2 | USA100/800 | ST5-MRSA-II | − | II | t002 | ST5 | + |
| | C4000 | USA100/800 | ST225-MRSA-II | − | II | t004 | ST225 | + |
| | CMRSA-3 | Like EMRSA1/4/11 | ST241-MRSA-III | − | III | t037 | ST241 | + |
| | CMRSA-6 | Like EMRSA1/4/11 | ST239-MRSA-III | − | III | t037 | ST239 | + |
| | C1777 | Like EMRSA1/4/11 | ST239-MRSA-III | − | III | t037 | ST239 | + |
| | CMRSA-4 | USA200/EMRSA16 | ST36-MRSA-II | − | II | t018 | ST36 | + |
| | C23374 | USA200/EMRSA 16 | ST36-MRSA-II | − | II | t018 | ST36 | + |
| | CMRSA-5 | USA500 | ST8-MRSA-IVd | − | IVd | t064 | ST8 | + |
| | CMRSA-8 | EMRSA 15 | ST22-MRSA | − | NT | t022 | ST22 | + |
| | CMRSA-9 | | ST8-MRSA | − | NT | t008 | ST8 | + |
| | C74 | | ST5-MRSA-IVb | − | IVb | t1154 | ST5 | + |
| PVL(+) | H435 | | ST5-MRSA-II | + | II | t311 | ST5 | + |
| MRSA | | | | | | | | |
| | PMRSA-34 | | ST59-MRSA-III | + | III | t437 | ST59 | + |
| | MR37 | Like USA400 | ST1-MRSA-IVa | + | IVa | t175 | ST1 | + |
| | MR138 | | ST80-MRSA-IVa | + | IVa | t044 | ST80 | + |
| | H434 | Like USA1100 | ST30-MRSA IVc | + | IVc | t019 | ST30 | + |
| | PMRSA-29 | USA1100 | ST30-MRSA-IVc | + | IVc | t019 | ST30 | + |
| | C1538 | USA1100 | ST30-MRSA-IVc | + | IVc | t019 | ST30 | + |
| PVL(+) | MS02-W10 | USA800 | ST5-MSSA | + | N/A | t015 | ST5 | + |
| MSSA | | | | | | | | |
| | SA5 | | ST121-MSSA | + | N/A | Unnamed | ST121 | + |
| | SA112 | | ST121-MSSA | + | N/A | t645 | ST121 | + |
| | SA125 | | ST25-MSSA | + | N/A | t436 | ST25 | + |
| | SA134 | | ST22-MSSA | + | N/A | t005 | ST22 | + |
| | SA28 | | ST59-MSSA | + | N/A | t437 | ST59 | + |
| | MS03-B1 | | ST59-MSSA | + | N/A | t437 | ST59 | + |
| | SA3 | | ST30-MSSA | + | N/A | unnamed | ST30 | + |
| | H49 | | ST30-MSSA | + | N/A | t021 | ST30 | + |
| | SAF516 | | ST30-MSSA | + | N/A | t483 | ST30 | + |
| PVL(−) | S. epidermidis | | ACME (+) | | | | | + |
| MS-CoNS | (ATCC12228) | | | | | | | |
| PVL(−) | CNS99-PF5 | | ACME (−) | | | | | + |
| MS-CoNS | | | | | | | | |
| PVL(−) | CNS99-PF7 | | ACME (+) | | | | | + |
| MS-CoNS | | | | | | | | |
| PVL(−) | S. epidermidis | | ACME (+) | | | | | + |
| MR-CoNS | (GISE 12333) | | | | | | | |
| PVL(−) | CNS99-PF6 | | ACME (−) | | | | | + |
| MR-CoNS | | | | | | | | |
| PVL(−) | CNS99-PF8 | | ACME (+) | | | | | + |
| MR-CoNS | | | | | | | | |

| | | Genotype | | | | | |
|---|---|---|---|---|---|---|---|
| | | S. aureus | Methicillin | PVL | Phage | USA400 | USA300 |
| Strain | | specific | specific | specific | specific | specific | specific |
| Type | Strain | Nuc | mecA | PVL | MW1409 | MW756 | arcA |
| PVL(+) | CMRSA10 | + | + | + | + | − | + |
| USA300 | | | | | | | |
| | PMRSA-3 | + | + | + | + | − | + |
| | PMRSA-13 | + | + | + | + | − | + |
| | PMRSA-16 | + | + | + | + | − | + |
| | PMRSA-46 | + | + | + | + | − | + |
| | PMRSA-12 | + | + | + | + | − | + |
| PVL(−) | C2901 | + | + | − | − | + | − |
| USA400 | | | | | | | |
| | C2140 | + | + | − | − | + | − |
| | C6022 | + | + | − | − | + | − |
| PVL(+) | CMRSA-7 | + | + | + | + | + | − |
| USA400 | | | | | | | |
| | PMRSA-18 | + | + | + | + | + | − |
| | PMRSA-50 | + | + | + | + | + | − |
| | C10687 | + | + | + | + | + | − |
| PVL(−) | CMRSA-1 | + | + | − | − | − | − |
| MRSA | | | | | | | |
| | CMRSA-2 | + | + | − | − | − | − |
| | C4000 | + | + | − | − | − | − |
| | CMRSA-3 | + | + | − | − | − | − |
| | CMRSA-6 | + | + | − | − | − | − |
| | C1777 | + | + | − | − | − | − |
| | CMRSA-4 | + | + | − | − | − | − |
| | C23374 | + | + | − | − | − | − |
| | CMRSA-5 | + | + | − | + | − | − |
| | CMRSA-8 | + | + | − | − | − | − |
| | CMRSA-9 | + | + | − | − | − | − |
| | C74 | + | + | − | − | − | − |
| PVL(+) | H435 | + | + | + | − | − | − |
| MRSA | | | | | | | |
| | PMRSA-34 | + | + | + | − | − | − |
| | MR37 | + | + | + | − | − | − |
| | MR138 | + | + | + | − | − | − |
| | H434 | + | + | + | − | − | − |
| | PMRSA-29 | + | + | + | − | − | − |
| | C1538 | + | + | + | − | − | − |
| PVL(+) | MS02-W10 | + | − | + | − | − | − |
| MSSA | | | | | | | |
| | SA5 | + | − | + | − | − | − |
| | SA112 | + | − | + | − | − | − |
| | SA125 | + | − | + | − | − | − |
| | SA134 | + | − | + | − | − | − |
| | SA28 | + | − | + | − | − | − |
| | MS03-B1 | + | − | + | − | − | − |
| | SA3 | + | − | + | − | − | − |
| | H49 | + | − | + | − | − | − |
| | SAF516 | + | − | + | + | − | − |
| PVL(−) | S. epidermidis | − | − | − | − | − | + |
| MS-CoNS | (ATCC12228) | | | | | | |
| PVL(−) | CNS99-PF5 | − | − | − | − | − | − |
| MS-CoNS | | | | | | | |
| PVL(−) | CNS99-PF7 | − | − | − | − | − | + |
| MS-CoNS | | | | | | | |
| PVL(−) | S. epidermidis | − | + | − | − | − | + |
| MR-CoNS | (GISE 12333) | | | | | | |
| PVL(−) | CNS99-PF6 | − | + | − | − | − | − |
| MR-CoNS | | | | | | | |
| PVL(−) | CNS99-PF8 | − | + | − | − | − | + |
| MR-CoNS | | | | | | | |

^{a} spa types: t002 (TJMBMDMGMK), t004 (A2AKEEMBKB), t005 (TJEJNCMOMOKR), t008 (YHGFMBQBLO), t018 (WGKAKAOMQQQ), t019 (XKAKAOMQ), t021 (WGKAKAOMQ), t022 (TJEJNF2MNF2MOMOKR), t037 (WGKAOMQ), t044 (UJGBBPB), t064 (YHGCMBQBLO), t105 (TJMBMDMMK), t128 (UJJFKBPE), t175 (UJFKKPFKPE), t311 (TJMBDMGMK), t436 (ZFGU2DM), t437 (ZDMDMOB), t483 (WGKKAKAOMQQQQ), t645 (I2Z2EGMJH2M), t1154 (TDMGMK), SA5 unnamed (I2H2M), SA3 unnamed (I2H2M).
^{b} MLST profiles: ST1 (1-1-1-1-1-1-1), ST5 (1-4-1-4-12-1-10), ST8 (3-3-1-1-4-4-3), ST22 (7-6-1-5-8-5-6), ST25 (4-1-4-1-5-5-4), ST30 (2-2-2-2-6-3-2), ST36 (2-2-2-2-3-3-2), ST45 (10-14-8-6-10-3-2), ST59 (19-23-15-2-19-20-15), ST80 (1-3-1-14-11-51-10), ST121 (6-5-6-2-7-14-5), ST225 (1-4-1-4-12-25-10), ST239 (6-5-6-2-7-14-5), ST241 (2-3-1-1-4-4-30), PMRSA-13 new (3-3-1-4-4-4-3).

TABLE 3
M-PCR sensitivity in representative strains

| Strain | | | Molecular | | SCC | SPA | ST | |
|---|---|---|---|---|---|---|---|---|
| Type | Strain | PFGE Type | Type | PVL | mec | Type | Type | PCR |
| PVL(+) | CMRSA10 | USA300 (0114) | ST8-MRSA-IVa | + | IVa | t008 | ST8 | M-PCR |
| USA300 | | | | | | | | |
| | | | | | | | | Single target |
| | | | | | | | | PCR |
| PVL(+) | CMRSA-7 | USA400 | ST1-MRSA-IVa | + | IVa | t128 | ST1 | M-PCR |
| USA400 | | | | | | | | |
| | | | | | | | | Single target |
| | | | | | | | | PCR |

| | Sensitivity (CFU/PCR) for the following genes | | | | | | |
|---|---|---|---|---|---|---|---|
| | Staph. sp. | S. aureus | Methicillin | PVL | Phage | USA400 | USA300 |
| Strain | Specific | specific | specific | specific | specific | specific | specific |
| Type | 16srRNA | Nuc | mecA | PVL | MW1409 | MW756 | arcA |
| PVL(+) | 6 × 10⁶ | 6 × 10⁶ | 6 × 10⁶ | 6 × 10⁶ | 6 × 10⁶ | 6 × 10⁶ | N/A |
| USA300 | | | | | | | |
| | 6 × 10⁵ | 6 × 10⁵ | 6 × 10⁵ | 6 × 10⁴ | 6 × 10⁵ | 6 × 10⁵ | N/A |
| PVL(+) | 6 × 10⁶ | 6 × 10⁶ | 6 × 10⁶ | 6 × 10⁶ | 6 × 10⁶ | N/A | 6 × 10⁶ |
| USA400 | | | | | | | |
| | 6 × 10⁵ | 6 × 10⁵ | 6 × 10⁵ | 6 × 10⁵ | 6 × 10⁵ | N/A | 6 × 10⁵ |

TABLE 4
Validation of M-PCR assay in clinical isolates

| | | | | | Staphylococcus | | | | | |
|---|---|---|---|---|---|---|---|---|---|---|
| | | | No | Staphylococcus | aureus | Methicillin | PVL | Phage | USA400 | USA300 |
| Strain | PFGE | Number | indifferent | specific | specific | specific | specific | specific | specific | specific |
| Type | profile | Tested | types | 16s rRNA | nuc | mecA | PVL | MW1409 | MW756 | arcA |
| PVL(+) | USA300 | 54 | 54 | + | + | + | + | + | − | + |
| USA300* | | | | | | | | | | |
| PVL(+) | USA400 | 17 | 17 | + | + | + | + | + | + | − |
| USA400* | | | | | | | | | | |
| PVL(−) | USA400 | 35 | 35 | + | + | + | − | − | + | − |
| USA400* | | | | | | | | | | |
| PVL(−) | Non- | 74 | | | | | | | | |
| MRSA* | USA300 | | | | | | | | | |
| | Non- | | 74 | + | + | + | − | − | − | − |
| | USA400 | | | | | | | | | |
| PVL(−) | ND | 255 | 244 | + | + | + | − | − | − | − |
| MRSA | | | 5 | + | + | + | − | + | − | − |
| | | | 6 | + | + | + | − | − | + | − |
| PVL(+) | ND | 1 | 1 | + | + | + | + | − | − | − |
| MRSA | | | | | | | | | | |
| PVL(−) | ND | 258 | 253 | + | + | − | − | − | − | − |
| MSSA | | | 5 | + | + | − | − | + | − | − |
| PVL(−) | ND | 214 | 139 | + | − | − | − | − | − | − |
| meS CoNS | | | 75 | + | − | − | − | − | − | + |
| PVL(−) | ND | 225 | 132 | + | − | + | − | − | − | − |
| meR CoNS | | | 93 | + | − | + | − | − | − | + |

*Assigned based on PFGE profile and PVL genes. Total = 1133

### Example 5

### Results

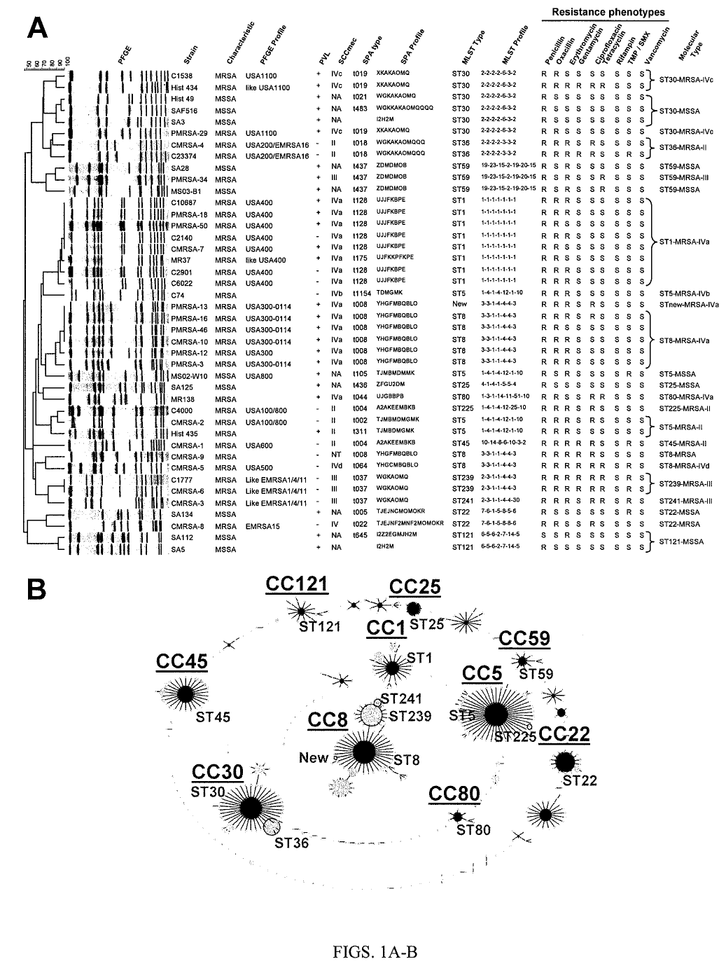

Use of well-representative control strains for validation of the new M-PCR assay. The use of a large collection of representative control strains have served as the basis for development and validation of the new M-PCR assay. The chosen representative strains (isolates) from the inventors' collection had undergone detailed phenotypic, genotypic and molecular characterization, including determination of their antimicrobial resistance profiles, carriage of PVL and other genes, PFGE fingerprints, SCCmec typing, agr typing, spa typing, MLST and eBURST analyses. There were 42 strains (isolates) of S. aureus, including 6 PVL(+) USA300, 3 PVL(−) USA400, 4 PVL(+) USA400, 12 other PVL(−) MRSA, 7 other PVL(+) MRSA, and 10 PVL(+) MSSA (Table 2). They varied in their phenotypic and genotypic characteristics (Table 2; FIG. 1A), and represented 10 major clonal complex (CC) groups found in the worldwide MLST collection (FIG. 1B). Since the ACME element (cassette) is widely distributed in CoNS isolates, the inventors also included 6 representative strains (isolates) from CoNS, including 2 ACME(+) MS (methicillin-susceptible)-CoNS, 1 ACME(−) MS-CoNS, 2 ACME(+) MR (methicillin-resistant)-CoNS, and 1 ACME(−) MR-CoNS (Table 2).

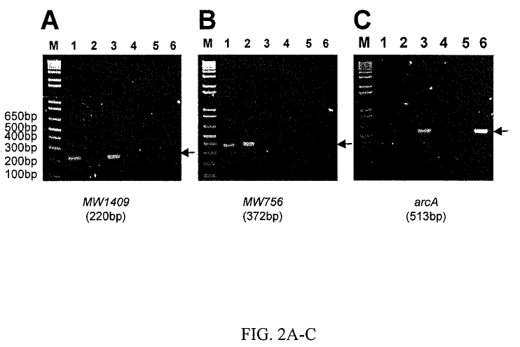

Phage- and strain-specific primer design and validation. The φSa2mw/usa Phage-specific, and USA300 and USA400 strain-specific primers were designed following extensive comparisons of all staphylococcal genomes currently available in the GenBank database. The genes coding for PVL are carried on a select number of prophages, including φPVL (Kaneko et al., 1998), φPV83 (Zou et al., 2000), φSLT (Narita et al., 2001), φSa2mw (Baba et al., 2002), φSa2usa (Diep et al., 2006) and, as recently reported, φ108PVL (Ma et al., 2006). The φSa2mw phage from MW2 shows remarkable sequence homology to φSa2usa from USA300 (Diep et al., 2006), and both differ in composition from the other phages (Ma et al., 2006). Analysis of the complete phage sequence led to identification of the region unique to the USA400 and USA300 φSa2mw/φSa2usa prophages. Primers targeting gene MW1409 within these unique region were subsequently designed. Screening the above 48 control strains (Table 2), as well as a random set of clinical isolates including 10 PVL(+) USA300, 10 PVL(+) USA400, 10 PVL(−) USA400, 10 other MRSA, 10 MSSA, and 10 CoNS indicated that all 16 PVL(+)USA300 and 14 PVL(+)USA400 were positive for this target, while the remaining 74 strains/isolates were negative (FIGS. 2A and 2B).

Differentiation of the strain USA300 from the strain USA400 and other staphylococcal strains was achieved by targeting regions unique to each strain. Strain MW2 contains several genomic elements which contribute to its virulence, however, genomic island νSa3 was reported to be unique to this strain (Baba et al., 2002). Analysis of this 14 kb element identified several short regions specific to the USA400 genome, from which primers targeting MW756 were designed. Screening of the control strains and the random set of clinical isolates with this primer pair yielded the expected amplified product (372 bp) for all 14 PVL(+) and 13 PVL(−) USA400 isolates and negative results for the remaining 61 strains/isolates (FIG. 2C). For USA300 the ACME complex (containing the arc gene cluster coding for a complete set of enzymes involved in the arginine deiminase pathway as well as an opp oligopeptide permease operon) was selected. While all S. aureus strains carry on their chromosome a native arc gene complex, An Diep et al. (Diep et al., 2006) have reported finding a modified ACME complex solely in S. epidermidis, S. capitis and USA300. arcA codes for the central enzyme in this pathway, arginine deiminase, and was found to be specific to the USA300 and S. epidermidis ATCC12228 genomes by sequence analysis. After screening the control strains and the random set of clinical isolates, the inventors noted that all 16 PVL(+)USA300 were positive for arcA, while the remaining other S. aureus strains were negative. They also noted that S. epidermidis strain GISE 12333 (similar to ATCC1228 as shown in Table 2), as well as 38.3% of the CoNS clinical isolates (unknown species; Table 4) were positive (FIG. 2D).

A new M-PCR assay for typing MRSA isolates and identifying USA300 and USA400 strains. The inventors developed a new multiplex PCR assay capable of not only identifying USA300 and USA400 strains, but of also simultaneously discriminating S. aureus from CoNS, methicillin-sensitive (MS) from methicillin-resistant (MR) staphylococci, and PVL(+) from PVL (−) strains. The assay specifically involved targeting the Staphylococcus genus-specific 16s rRNA gene sequence, the S. aureus specific nuc gene, the methicillin resistance determinant mecA, the PVL genes, the phage specific gene MW1409, the USA400 specific genomic island gene (MW756) and the USA300 specific ACME cassette gene (arcA). The Staphylococcus genus-specific primers are specific to the staphylococcal 16s rRNA gene and identify bacteria to the genus level, as well as serve as an internal control for this assay. Staphylococcus aureus can be distinguished from CoNS with the S. aureus species-specific nuc gene in combination of the Staphylococcus genus-specific primers, while discrimination between methicillin-sensitive and methicillin-resistant staphylococci (including MRSA from MSSA and MR-CoNS from MS-CoNS) is accomplished with primers targeted to mecA, the determinant of methicillin resistance. PVL is postulated to be on of the major virulence determinants of CA-MRSA and is found in both USA300 and PVL(+) USA400 isolates. The PVL genes are carried on a select number of prophages, however, this assay specifically targeted the prophages of φSa2mw from USA400 and φSa2usa from USA300. Not only is the phage specific primer pair indicative of the presence of the phage in USA300 or USA400, it also provides information as to weather these phages are present in other staphylococcal strains. The genomic island νSa3 is unique to USA400 strain MW2 while the ACME complex is unique to USA300 strain FPR3757. As such, primers targeted to MW756 on νSa3 of MW2 and arcA on the ACME complex of USA300 were chosen to discriminate between these otherwise similar strains and other Staphylococcus strains.

Single target PCR reactions were performed prior to multiplex optimization to ensure that the individual primer pairs were adequate for amplification of all loci under the specified conditions. Canadian epidemic MRSA control strains CMRSA-7 (USA400) and CMRSA-10 (USA300) were used to verify that the amplification reactions yielded expected product sizes of 147, 220, 279, 372, 433, 513 and 756 bp for mecA, MW1409, nuc, MW756, PVL, arcA and 16s rRNA, respectively. Primer concentrations were adjusted to yield a final M-PCR assay showing distinct bands of the correct molecular size, easily recognizable in a 2% agarose gel stained with ethidium bromide (FIG. 3).

Sensitivity of M-PCR. The sensitivity of the M-PCR assay versus single target PCR was examined with two representative control strains, CMRSA10 (USA300, known to harbor 6 of the 7 target genes by virtue of lacking the USA400 strain-specific MW756 sequence) and CMRSA7 (USA400, known to harbor 6 of the 7 target genes by virtue of lacking the USA300 strain-specific arcA gene). This assay was capable of detecting, with reproducibility, a band in ethidium bromide-stained gels at dilutions corresponding to 6×10⁶ CFU per PCR reaction for all 7 target genes (Table 3). This sensitivity is quite compatible with the single target PCR assay (6×10⁴⁻⁵) (Table 3), suggesting that the M-PCR assay is sufficiently robust.

Validation of M-PCR Assay. The new multiplex PCR assay was validated using 42 well-characterized staphylococcal control strains with known PVL, mecA, SCCmec, agr, spa, MLST and PFGE typing and other phenotypic information, as well as 6 CoNS control strains (Table 2). The assay was capable of accurately and reproducibly discriminating USA300 strain from USA400 strain or other MRSA, and MRSA from MSSA, and simultaneously detecting PVL genes and φSa2mw/φSa2usa phage in 100% concordance with phenotypic and genotypic features in all these control strains (Table 2). There were 7 PVL(+) MRSA and 9 PVL(+) MSSA strains, which, belonging to non-USA300 and non-USA400 strains with well-diversified genomic backgrounds according to genotypic and PFGE profiles, were positive for the PVL genes but negative for the φSa2mw/φSa2usa phage specific PCR product of MW1409 (FIGS. 1A-B; Table 2), suggesting that PVL genes in these strains may be carried by other phage or plasmids rather than φSa2mw/φSa2usa phages. More interestingly, there were two strains (non-USA300 and non-USA400 PFGE profiles), one PVL(−) MRSA (CMRSA-5; PFGE profile USA500, MLST type ST8, spa type t064, SCCmec type IVd and multidrug-resistant phenotypes) and one PVL (+) MSSA (SAF516; non classified PFGE profile, MLST type ST30, spa type t483, and β-latam only resistant phenotypes) (FIGS. 1A-B and Table 2), which were positive for the phage specific PCR product of MW1409. Because this primer pair is specific for the PVL− bearing phage φSa2mw/φSa2usa in USA300 and USA400, it suggests that variations of these particular phages can also be present in other S. aureus strains than USA300 and USA400, both with or without the PVL genes.

Applicability and accuracy of M-PCR. To address the applicability and accuracy of the M-PCR assay, the inventors further applied our M-PCR assay to test a total of 1133 local clinical MRSA isolates randomly selected from the inventors' Calgary frozen clinical isolate stock collection for the 18 year period from 1989 to 2006 (Table 4). All of the isolates had undergone PVL, mecA and SCCmec typing (applied to only MRSA isolates) and 180 of them had previous PFGE data available. The inventors were able to accurately identify and classify all strains with available PFGE data including 54 PVL(+) USA300, 17 PVL(+) USA400, 35 PVL(−) USA400, and 74 PVL(−) non-USA300 or non-USA400 MRSA (Table 4).

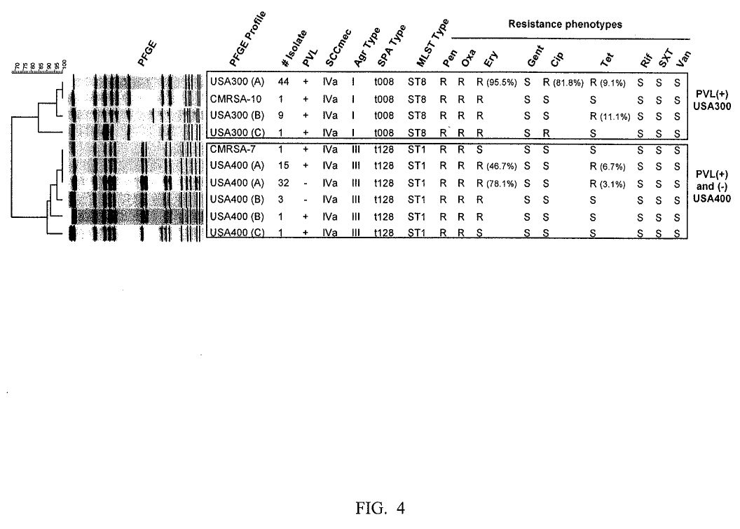

There were 3 PFGE patterns, A, B and C, identified in these 54 USA300 isolates (FIG. 4). Pattern A, which was indistinguishable from the USA300 control strain CMRSA10, was noted for 44 isolates (81.5%). Nine (16.7%) isolates shared a Pattern B PFGE profile while only 1 isolates (1.9%) had a pattern C profile. However, all of these isolates were PVL positive, carried the SCCmec type IVa element, and shared the same MLST ST8 profile (3-3-1-1-4-4-3), spa type t008 motif (YHGFMBQBLO) and agr type I (FIG. 4). All isolates were resistant to β-lactams, but uniformly susceptible to all other antibiotics except erythromycin (96.3% resistant), ciprofloxacin (68.5% resistant) and tetracycline (9.3% resistant) (FIG. 4). The inventors M-PCR assay accurately identified all these varied USA300 isolates (Table 4).

In those 52 USA400 isolates, there were 17 PVL (+) and 35 PVL (−). Both PVL (+) and PVL (−) isolated shared 3 PFGE patterns, with 47 (90.4%; 15 PVL+ vs 32 PVL−), 4 (7.7%; 1 PVL+ vs 3 PVL−) and 1(1.9%; PVL+) with Pattern A, B and C, respectively (FIG. 4). The dominant pattern was Pattern A, which was indistinguishable from the USA400 control strain CMRSA7 (same as PVL(+) USA400/W2 strain (Mulvey et al., 2005)). Regardless of the presence of PVL gene and the PFGE pattern, all of these isolates carried the SCCmec type IVa element, and shared the same MLST ST1 profile (1-1-1-1-1-1-1), spa type t128 motif (UJJFKPE) and agr type III. All isolates were resistant to β-lactams, but uniformly susceptible to all other antibiotics except erythromycin (69.2% resistant) and tetracycline (3.8% resistant) (FIG. 4). Again, the assay accurately identified all these PVL(+) and (−) varied USA400 isolates (Table 4).

The inventors were also able to clearly classify the remaining randomly chosen strains, including 514 Staphylococcus aureus and 439 CoNS isolates (Table 4). Once again, they noted that 10 (1.9%) of the isolates, including 5 PVL(−) MRSA and 5 PVL(−) MSSA, were positive for the phage specific gene yet did not belong to either USA300 or USA400 (Table 4). There was also 1 MRSA isolate that was positive for the PVL genes, but negative for the φSa2mw/φSa2usa phage specific gene (Table 4). Six of the random chosen MRSA isolates with no initial PFGE data available were identified as PVL(−) USA400 and later confirmed to have a PFGE pattern matching USA400 (Table 4). Among 439 CoNS isolates tested, there was a 100% concordance with phenotypic susceptibility to methicillin, with 214 being MS-CoNS and 225 being MR-CoNS. Of the methicillin sensitive isolates, 75 (35.0%) of them were positive for arcA, while 93 (41.3%) of the methicillin resistant CoNS were arcA positive. None of the CoNS isolates tested in this study carried PVL, φSa2mw/usa phage and USA400 specific MW756 genes (Table 4). None of the CoNS isolates tested in this study were identified to the species level; therefore determining if the arcA positive CoNS were restricted to S. epidermidis is unknown.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### C. References

The following references, to the extent that they provide exemplary procedural or other details supplementary to those set forth herein, are specifically incorporated herein by reference.
U.S. Pat. No. 4,659,774
U.S. Pat. No. 4,816,571
U.S. Pat. No. 4,959,463
U.S. Pat. No. 5,141,813
U.S. Pat. No. 5,264,566
U.S. Pat. No. 5,428,148
U.S. Pat. No. 5,554,744
U.S. Pat. No. 5,574,146
U.S. Pat. No. 5,602,244
Ayliffe, Clin. Infect. Dis., 24:S74-9, 1997.
Baba et al., Lancet., 359(9320):1819-1827, 2002.
Barber, J. Clin. Pathol., 14:385-393, 1961.
Beaucage, Methods Mol. Biol., 20:33-61, 1993.
Begier et al., Clin Infect Dis., 39(10):1446-1453, 2004.
Beilman et al., Surg Infect (Larchmt)., 6(1):87-92, 2005.
Bratu et al., Emerg. Infect. Dis., 11(6):808-813, 2005.
Chalumeau et al., Clin. Infect. Dis., 41(3):e29-30, 2005.
Conly et al., Can. J. Infect. Dis. Med. Microbiol., 16:109, 2005.
Crossley et al., J. Infect. Dis., 139:273-279, 1979.
Diep et al., Lancet., 367(9512):731-739, 2006.
Embil et al., Infect. Control Hosp. Epidemiol., 15:646-651, 1994.
Enright et al., J. Clin. Microbiol., 38(3):1008-1015, 2000.
Feil et al., Curr. Opin. Microbiol., 7(3):308-313, 2004.
Francis et al., Clin. Infect. Dis., 40(1):100-107, 2005.
Gilbert et al., Can. J. Infect. Dis. Med. Microbiol., 16:108, 2005.
Gilbert et al., CMAJ, 175(2):149-154, 2006.
Gillam et al., J. Biol. Chem., 253:2532-2539, 1978.
Hackbarth & Chambers, Antimicrob. Agents Chemother., 33(7):995-999, 1989.
Harbarth et al., Emerg. Infect. Dis., 11(6):962-965, 2005.
Harmsen et al., J. Clin. Microbiol., 41(12):5442-5448, 2003.
Holmes et al., J. Clin. Microbiol., 43(5):2384-2390, 2005.
Hussain et al., J. Clin. Microbiol., 38:752-754, 2000.
Issartel et al., Clin. Microbiol., 43(7):3203-3207, 2005.
Itakura et al., J. Biol. Chem., 250:4592 1975.
Ito et al., Antimicrob. Agents Chemother., 45:1323-1336, 2001.
Ito et al., Antimicrob. Agents Chemother., 48:2637-2651, 2004.
Jevons, British Med. J, 1:124-125, 1961.
Kaneko et al., Gene, 215(1):57-67, 1998.
Kazakova et al., N. Engl. J. Med., 352(5):468-475, 2005.
Khorana, Science, 203(4381):614-625, 1979.
Lina et al., Clin. Infect. Dis., 29(5):1128-1132, 1999.
Linde et al., Eur. J. Clin. Microbiol. Infect. Dis., 24(6):419-422, 2005.
Lindsay and Holden, Trends Microbiol., 12:378-385, 2004.
Livermore, Int. J. Antimicrob. Agents, 16(1:)S3-10, 2000.
Ma et al., Emerg. Infect. Dis., 11(6):973-976, 2005.
Ma et al., Antimicrob. Agents Chemother., 46:1147-1152, 2002.
McClure et al., J. Clin. Microbiol., 44(3):1141-1144, 2006.
Mulvey et al., Emerg. Infect. Dis., 11(6):844-850, 2005.
Mulvey et al., J. Clin. Microbiol., 39(10):3481-3485, 2001.
Murray, In: Manual of Clinical Microbiology, 8^{th} Ed., American Society for Microbiology Press, Washington, 2003.
Naas et al., J. Hosp. Infect., 61(4):321-329, 2005.
Narita et al., Gene, 268(1-2):195-206, 2001.
Pan et al., Clin. Infect. Dis., 37(10):1384-1388, 2003.
Panlilio et al., Infect. Control Hosp. Epidemiol., 13:582-586, 1992.
Robert et al., Clin. Microbiol. Infect., 11(7):585-587, 2005.
Said-Salim et al., J. Clin. Microbiol., 43(7):3373-3379, 2005.
Sambrook et al., In: Molecular cloning: a laboratory manual, 2^{nd} Ed., Cold Spring Harbor Laboratory Press, Cold Spring Harbor, N.Y., 1989.
Shopsin et al., J. Clin. Microbiol., 37(11):3556-3563, 1999.
Shortle, Gene, 22(2-3):181-189, 1983.
Simor et al., J. Infect. Dis., 186:652-660, 2002.
Spratt et al., FEMS Microbiol. Lett., 241(2):129-134, 2004.
Tenover et al., J. Clin. Microbiol., 33(9):2233-2239, 1995.
Tenover et al., J. Clin. Microbiol., 44(1):108-118, 2006.
Vandenesch et al., Emerg. Infect. Dis., 9(8):978-984, 2003.
Vandenesch et al., Emerg. Infect. Dis., 9:978-984, 2003.
Voss et al., Eur. J. Clin. Microbiol. Infect. Dis., 13:50-55, 1994.
Vourli et al., Euro. Surveill., 10(5):78-79, 2005.
Wannet et al., J. Clin. Microbiol., 42(7):3077-3082, 2004.
Wannet et al., J. Clin. Microbiol., 43(7):3341-3345, 2005.
Witte et al., Eur. J. Clin. Microbiol. Infect. Dis., 24(1):1-5, 2005.
Wylie and Nowicki, J. Clin. Microbiol., 43(6):2830-2836, 2005.
Zhang et al., J. Clin. Microbiol., 42:4947-4955, 2004.
Zhang et al., J. Clin. Microbiol., 43(10):5026-5033, 2005.

What is claimed is:

1. A method for identifying a methicillin-resistant Staphylococcal aureus (MRSA) bacterium in a sample comprising:
(a) obtaining a biological sample suspected of containing a MRSA bacterium;
(b) subjecting nucleic acids from said sample to multiplex PCR targeting the following genes: 16s rRNA, arcA, lukS/F-PV, MW756, nuc, MW1409, mecA and MW1438;
(c) detecting amplification products resulting from said multiplex PCR; and
(d) comparing the detected amplification products from step (c) with those predicted for known MRSA and/or non-MRSA bacteria,
wherein an amplification product profile similar or identical to that observed for known MRSA bacteria identifies said sample as comprising a MRSA bacterium.

2. The method of claim 1, wherein said assay further discriminates between USA300/USA400 and other MRSA strains.

3. The method of claim 1, wherein said assay further discriminates between USA300 and USA400.

4. The method of claim 1, wherein said biological sample is a clinical sample.

5. The method of claim 4, wherein said clinical sample is urine, blood, sputum, saliva, or pus.

6. The method of claim 1, wherein said biological sample is a culture sample.

7. The method of claim 6, wherein said culture sample is from a broth, a plate or membrane.

8. The method of claim 1, wherein said multiplex PCR is performed using one or more of the following primer pairs: SEQ ID NO:1 and SEQ ID NO:2; SEQ ID NO:3 and SEQ ID NO:4; SEQ ID NO:5 and SEQ ID NO:6; SEQ ID NO:7 and SEQ ID NO:8; SEQ ID NO:9 and SEQ ID NO:10; SEQ ID NO:11 and SEQ ID NO:12; SEQ ID NO:13 and SEQ ID NO:14; and/or SEQ ID NO:15 and SEQ ID NO:16.

9. The method of claim 8, wherein each of said multiplex PCR is performed using each of said primer pairs.

10. The method of claim 1, wherein detecting amplification products comprises electrophoretic separation and visualization of separated amplification products.

11. A kit comprising primer pairs for amplifying the following genes: 16s rRNA, arcA, lukS/F-PV, MW756, nuc, MW1409, mecA and MW1438, in one or more suitable containers.

12. The kit of claim 11, wherein said primer pairs are: SEQ ID NO:1 and SEQ ID NO:2; SEQ ID NO:3 and SEQ ID NO:4; SEQ ID NO:5 and SEQ ID NO:6; SEQ ID NO:7 and SEQ ID NO:8; SEQ ID NO:9 and SEQ ID NO:10; SEQ ID NO:11 and SEQ ID NO:12; SEQ ID NO:13 and SEQ ID NO:14; and SEQ ID NO:15 and SEQ ID NO:16.

13. The kit of claim 11, further comprising a buffer, diluent and/or excipient in at least one additional suitable container.

14. The kit of claim 11, further comprising a enzyme that directs polymerase chain reaction in at least one additional suitable container.

15. The kit of claim 11, further comprising at least one molecular weight standard or PCR substrate standard in at least one additional suitable container.


SEQUENCE LISTING (16 sequences)

SEQ ID NO: 1 - DNA, 23 bp, Artificial
  Synthetic primer
aactctgttattagggaagaaca

SEQ ID NO: 2 - DNA, 23 bp, Artificial
  Synthetic primer
ccaccttcctccggtttgtcacc

SEQ ID NO: 3 - DNA, 23 bp, Artificial
  Synthetic primer
gcagcagaatctattactgagcc

SEQ ID NO: 4 - DNA, 24 bp, Artificial
  Synthetic primer
tgctaacttttctattgcttgagc

SEQ ID NO: 5 - DNA, 31 bp, Artificial
  Synthetic primer
atcattaggtaaaatgtctggacatgatcca

SEQ ID NO: 6 - DNA, 27 bp, Artificial
  Synthetic primer
gcatcaagtgtattggatagcaaaagc

SEQ ID NO: 7 - DNA, 23 bp, Artificial
  Synthetic primer
tggttagctatgaatgtagttgc

SEQ ID NO: 8 - DNA, 22 bp, Artificial
  Synthetic primer
gtccatcctctgtaaattttgc

SEQ ID NO: 9 - DNA, 21 bp, Artificial
  Synthetic primer
gcgattgatggtgatacggtt

SEQ ID NO: 10 - DNA, 24 bp, Artificial
  Synthetic primer
agccaagccttgacgaactaaagc

SEQ ID NO: 11 - DNA, 21 bp, Artificial
  Synthetic primer
caaattttgaaaactttacgc

SEQ ID NO: 12 - DNA, 19 bp, Artificial
  Synthetic primer
tccaggattaaaagaagcg

SEQ ID NO: 13 - DNA, 21 bp, Artificial
  Synthetic primer
gtgaagatataccaagtgatt

SEQ ID NO: 14 - DNA, 22 bp, Artificial
  Synthetic primer
atgcgctatagattgaaaggat

SEQ ID NO: 15 - DNA, 20 bp, Artificial
  Synthetic primer
gaaaaaagtaatcggactgc

SEQ ID NO: 16 - DNA, 20 bp, Artificial
  Synthetic primer
gaaaaaagtaatcggactgc